(19) United States
(12) Patent Application Publication
(10) Pub. No.: US 2026/0070054 A1
(43) Pub. Date: Mar. 12, 2026

(54) CARD FOR STORING AND DISTRIBUTING NUCLEIC ACID MOLECULES

(71) Applicant: BIOSOLUTION DESIGNS, Frederick, MD (US)

(72) Inventor: Thomas REED, Frederick, MD (US)

(21) Appl. No.: 19/104,916

(22) PCT Filed: Aug. 25, 2023
(86) PCT No.: PCT/US2023/031112

§ 371 Date: Feb. 19, 2025

Related U.S. Application Data
(60) Provisional application No. 63/403,724, filed on Sep. 3, 2022.

(51) Int. Cl. B01L 3/00

(52) U.S. Cl. CPC B01L 3/5023; B01L 2200/185; B01L 2300/021; B01L 2300/069; B01L 2300/0816; B01L 2300/12; B01L 2300/126

(57) ABSTRACT

A card having a plurality of tabs comprising absorbent or binding material that can be spotted with nucleic acid solutions for storage and/or distribution to an end user, such as a customer purchasing the nucleic acids. An aqueous solution of nucleic acid molecules is spotted onto the absorbent or binding material, which is adhered to the tips of the tabs. Individual tabs may be cut or snapped off the card for recovery of the nucleic acid molecules. The card provides a convenient means of storage of small aliquots of the nucleic acid solution for weeks, months or years. Methods of assembling the cards and for spotting, storing and eluting the nucleic acid molecules are provided.

### FIELD OF THE INVENTION

The invention generally relates to compositions for storing nucleic acids in a format convenient for distribution to end users. The invention further relates to methods of making, using and storing on a card suitable for adsorbing nucleic acids.

### BACKGROUND

The uses of nucleic acid molecules have expanded in recent years, with worldwide distribution to researchers routinely required. However, methods of storage for distribution have changed little since the early days of nucleic acid isolation. Traditionally, specimens have been stored in aqueous solution at subzero temperatures of −20° C. or even −80° C. However, freezers take up space and have relatively limited capacities; they require an uninterrupted electricity supply to operate and can fail, sometimes with little warning. An extension of the need for electricity is that freezer storage is also not generally suitable for low resource environments. The aqueous solution for storage typically is buffered to prevent another source of damage to the nucleic acids. Unbuffered water is deleterious to nucleic acids due to pH swings during freezing/thawing, and ice crystals that form during freeze/thaw cycles can shear the molecules. Other agents frequently used in laboratory settings can be harmful to nucleic acids, particularly over time, so any storage solution must be free of these. For example, metal ions required for some enzymatic reactions can also allow damage to nucleic acid molecules. Inadvertent contamination of aqueous solutions can also cause damage. Furthermore, nucleic acids in aqueous solutions are typically shipped in cold packaging or even with dry ice, thus making distribution more expensive and wasteful.

Alternatively, nucleic acids, especially DNA, can be stored by spotting an aqueous solution containing the nucleic acid molecules of interest onto a small piece of filter paper, such as Whatmano® 3 mm chromatography paper (Merck KGaA; Germany) and allowing the spot to dry or desiccating the paper. Traditionally, a molecular biologist draws a circle on a piece of filter paper using a pencil and then spots aliquots of nucleic acids suspended in an aqueous buffer solution onto the circle. Each aliquot is allowed to dry before adding the next aliquot, until a desired amount of the nucleic acids is dried within the circle. In the dried or desiccated state, nucleic acids, DNA in particular, are very stable even at room temperature, especially if stored out of direct light. Dried labeled pieces of filter paper stored in boxes or other types of containers allow for long term storage at ambient temperature for many years under low humidity (less than ˜60%) conditions. Dried blood samples more than 20 years old are routinely viable for assays such as SNP genotyping or PCR assays. See Sjöholm et al. (Clin Chem. 53(8) 1401 (2007)) and Cassol et. al. (J Clin Micro. 30(12) 3039 (1992)). DNA plasmids are frequently shipped as dried specimens on filter papers and are considered to be generally safe methods of storage, but losses in DNA integrity over longer terms (>3 months) have been reported. See Murakami (Open Biotech 7(10) 2013). QIAcard® FTA® and the FTA® Elute Card are commercially available cards for room temperature collection, shipment, archiving and purification of nucleic acids. Each card has up to 4 circles for application of nucleic acid samples and a region for application of a single label for the card. The area of a dried sample on an FTA® Elute card must be punched to transfer up to four 3 mm punches to a secondary container for recovery and/or amplification of the nucleic acids.

DNA molecules in disaccharide sugar trehalose solution have also been successfully dried in multi-well format plates and found to be stable for at least two years at various storage temperatures. Alternatively, polyvinyl alcohol and various proprietary commercial reagents for dry DNA storage are known, such as Biomatrica® DNAstable® plates, which protect DNA at 56° C. and at room temperature longer than trehalose and PVA, especially for diluted samples. However, the DNA stored at −20° C. yielded longer sequence reads and stronger signal, indicating that temperature is a crucial factor for DNA quality which has to be considered especially for long-term storage in solutions dried on multi-well plates and similar substrates. See Ivanova and Kuzmina (Mol Ecol Resour. 2013 September; 13(5):890-8).

While spotting and labeling paper for nucleic acid storage is sufficient for managing a small number of nucleic acid species, there is still a need for producing large numbers of desiccated samples for commercial production and distribution in a format that is compact and trackable. It would also be advantageous to have a format suitable for barcoding or QR coding, such as that used for multi-well plates. It would also be an advantage to have a format that can store multiple nucleic acid species on a single piece of paper or card without concern about cross-contamination between the spots for each of the species, such as multiple plasmid DNA vectors having different inserts or other element(s). There is currently no product on the market that meets all these needs.

### SUMMARY OF THE INVENTION

The invention is a card comprising a thin substrate having tab sections formed by perforated or cut lines. The flat upper surface or bottom surface of the distal end of each tab has an absorbent material, such as paper, adhered on a surface of the substrate of the card, and is shaped to fit into a microtube. Nucleic acids can be applied to the absorbent material and placed in storage and/or distributed through the mail or other means of shipping to an end user.

In one embodiment, the invention is a card for storage of nucleic acid molecules, comprising a substrate of a suitable size and thickness, having perforations forming a plurality of detachable tabs, an absorbent material adhered to a flat upper surface or bottom surface of a distal end of each of the detachable tabs, and a proximal region on each of the detachable tab wherein a label may be applied. The absorbent material may be pretreated to remove or inhibit agents that would damage the nucleic acids. The absorbent material may be a porous paper that is adhered to the substrate with a non-toxic adhesive. The card may be labeled by printing directly on the substrate, or by application of an identifying label, such as a paper or plastic sticker. A label can identify the nucleic acid molecule that is absorbed on the absorbent material with the contents of each tab identified individually. A label identifying the card may also be printed or applied.

In another embodiment, the invention is a method of using a card for storage and/or distribution of nucleic acid molecules to an end user, comprising the steps of providing the solid substrate having a plurality of detachable tabs with absorbent material adhered to a flat upper surface or bottom surface of a distal end of each detachable tab; applying an aqueous solution comprising nucleic acid molecules to the absorbent material on at least one detachable tab; allowing the aqueous solution to dry; printing or applying a label identifying the nucleic acid molecules applied to the at least one detachable tab; sealing the card in packaging; and storing the packaged card under suitable conditions and/or distributing the packaged card to an end user (e.g., mailing, courier, etc.). Suitable conditions will depend upon the nature or identity of the nucleic acids spotted on the tabs. Typically, the conditions will be dry and shielded from light or UV irradiation and can include refrigeration, freezing or room temperature. When spotted with nucleic acids, dried and packaged, the card may be mailed or shipped to the end user.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIGS. 1A-1D show line drawings of various components of a nucleic acid storage card. FIG. 1A illustrates a thin plastic substrate 100 cut and printed and/or perforated (dashed lines 105) to form 16 pointed detachable tabs 110. FIG. 1B shows absorbent material 115 adhered to each tab 110. FIG. 1C shows a label 120 that identifies each tab with a unique QR code to provide information about nucleic acids to be spotted on the tabs of the card. FIG. 1D shows an assembled card identified with a product label 120 with QR codes to identify individual tabs.

FIG. 2 shows line drawings of nucleic acid storage cards having various configurations, including 20 tabs, 10 tabs and 5 tabs.

FIG. 3 shows a line drawing of a detached tab 110 having absorbent material 115 adhered to the distal tip and a microcentrifuge tube 20 for recovery of nucleic acids from the tab 110. Nucleic acids in an aqueous fluid with a neutral blue dye are spotted on the absorbent material 115 and dried. The detached tab 110 is placed in microcentrifuge tube 205 containing a suitable volume of buffered elution fluid. The change from clear to blue is an indicator that the dye and nucleic acids have transferred from the tab into the elution buffer 210.

FIG. 4 shows an exemplary card having one row of detachable tabs with an absorbent paper adhered to the distal shaped end of each tab. Each tab has been spotted with a solution of plasmid DNA to which a neutral blue dye has been added. One tab has been detached and placed into a microcentrifuge tube. Each tab is labeled with an identifying QR code providing a link to a description of the plasmid DNA. The card is also labeled with a QR code with information encoded that describes the contents of the card. As indicated on the card, the approximate size of the card may be 3.5″ wide and 2.5″ high.

FIG. 5 shows the intensity of color of eluent following 2 hours of elution from tabs spotted with nucleic acid solutions with 2×, 5× or 10× concentrations of dyes. 4A shows xylene cyanol, 4B shows bromophenol blue and 4C shows cresol red.

FIG. 6 shows bacterial growth of E. coli transformed with plasmid DNA eluted from tabs, as shown in FIG. 4. Xylene cyanol (XYLENE), which is toxic to E. coli, shows that no growth was observed. E. coli transformed with plasmids spotted with bromophenol blue (BROMO) or cresol red (CRESOL) and eluted for 2 hours showed similar amounts of growth.

FIG. 7 shows growth of E. coli transformed with plasmids successfully eluted in 120 μl of 1×TE buffer for 6A, 2 hours or 6B, 24 hours.

FIG. 8 shows the PCR products of DNA templates and PCR primers stored on a card at room temperature for 14 days, eluted and amplified. L=1 kb ladder.

### DETAILED DESCRIPTION

The following descriptions and examples illustrate some exemplary embodiments of the disclosed invention in detail. Those of the skill in the art will recognize that there are numerous variations and modifications of this invention that are encompassed by its scope. Accordingly, the description of a certain exemplary embodiment should not be deemed to limit the scope of the present invention.

As used herein, the term “nucleic acids” refers to RNA, mRNA, genomic DNA, plasmid DNA, synthetic DNA or RNA, siRNA, and oligonucleotides.

As used herein, the phrase “nucleic acid-protective properties” refers to properties that inhibit agents, compounds, enzymes and/or environmental forces that damage nucleic acids. For example, water absorbed from a humid environment can damage nucleic acids over time. The presence of nucleases in a nucleic acid solution or in materials of the invention can also cause damage. Nucleic acids should also be protected from metal ions since these may promote activity of contaminating enzymes. Thus, nucleic acid-protective agents may include but are not limited to metal ion chelators, nuclease inhibitors and/or desiccants.

One embodiment of the invention is a flat card comprising a solid substrate. The substrate may comprise a semi-rigid material that is capable of providing structural support to a test strip platform in which it may be incorporated. The substrate may also be referred to herein as a card backer. The substrate may be a material such as plastic (e.g., polyethylene (PET), polyvinylchloride (PVC), polyethylene terephthalate (PETG), polyimide, polycarbonate, polystyrene) ceramic, glass, paper or plastic-paper laminate or a combination of any of these. The material should be of a thickness that allows bending or cutting to detach the individual tabs and is typically in the range of 1-30 mil. A mil is a measurement that equals one-thousandth of an inch, or 0.001 inch. Most human hair is one-thousandth of inch, or 0.001 inch. The most common size in the thickness rating for plastic sheeting is 6 mil. This is 6-thousandths of an inch, or 0.006 inch. Generally, the thicker the plastic, the stronger it is. In some embodiments, the substrate may be made of metal. The substrate may be pre-treated to allow printing to obtain accurate color representation on the substrate.

The card has a plurality of tab sections formed by perforated or cut lines. The distal end of each tab has an absorbent material adhered on the flat upper surface. The proximal area of each tab can be used to apply a label or bar code. The card can be of any suitable size, but typically ranges from approximately the size of a credit card to a 3″×5″ note card. The tabs may be arranged in parallel rows that are perpendicular to the long edge of the card with absorbent material applied to tabs along one long edge, and an area for application of labels along the opposite long edge. A card may comprise tabs arranged along both long edges of the card with a central spine region where identifying labels can be applied. The distal ends of the tabs may be shaped to allow for insertion into a microcentrifuge tube or well of a multi-well plate. For example, the distal end may be tapered, pointed or rounded. In one embodiment, the distal end is square. The shape may be tailored to fit a particular type or style of tube. An advantage of the card of the invention is that the design prevents contamination from spots applied to an adjacent region. The paper-based cards of the prior art do not have a physical barrier preventing cross-contamination from wicking or have the potential to carry over nucleic acids on hole punch if not appropriately cleaned. In contrast, the card of the invention creates a physical barrier between tabs by eliminating absorbent contiguous material and provides a single, well-defined absorbent pad for binding of nucleic acids on a substrate of non-absorbent material. Additionally, the tab is designed for easy placement and removal from the elution tube by having the plastic tab near the top of the tube, distal from the absorbent pad, and readily grabbed with forceps or other means commonly found in laboratories.

The absorbent material may be treated to remove or inhibit agents that would damage the nucleic acids. The treatment may be a treatment applied prior to adhering the absorbent material to the substrate, i.e., a pretreatment, or it may be applied after the absorbent material is adhered to the substrate, i.e. as a spray or other liquid application to the absorbent material and substrate. Various agents, compounds or inhibitors have nucleic acid protective properties, making them well suited for this application. For example, the absorbent material may be treated with a chelator to bind metal ions that would otherwise activate contaminating enzymes and damage nucleic acids applied to the absorbent material, particularly over extended periods of time. Chelators include but are not limited to calcium disodium ethylenediamine tetraacetic acid (CaNa₂EDTA), ethylenediamine tetraacetic acid (EDTA), murexide, dimercaptol, desferrioxamine, deferoxamine, enterobactin, and calbindins. Other agents that prevent damage are inhibitors of nucleases, such as DNases, which degrade DNA, or RNases, which degrade RNA. On the other hand, one embodiment of a DNA storage card comprises absorbent material treated with DNase-free RNase to eliminate the possibility of RNA contamination. Use of water or other aqueous solutions treated with diethyl pyrocarbonate (DEPC), diethyl dicarbonate, diethyl oxydiformate or ethoxyformic acid anhydride is another protective treatment for RNA. Yet another example is protective treatment is with a desiccant, so that contact of the nucleic acids with water or humidity is minimized. Any of these treatments may be combined to provide protection and to extend the useful storage time of the nucleic acids.

Absorbent material is applied within a delineated area of the distal end of each tab to form an application region for nucleic acids. The application region of each tab may be smaller than the distal end of the tab to physically isolate each of the application regions on a card from each other, particularly if the detachable tabs are straight sided rather than tapered. The absorbent material may be a filter paper, such as an acid-free cotton paper. Many types and brands of absorbent paper are well known in the art, including Whatman® 3 mm chromatography paper (Merck KGaA; Germany). For example, a Whatman® No. 1 paper having a thickness of 180 μm or 0.18 mm works well for this purpose. Others that are suitable include Hamilco white cardstock, which is a heavy weight 100 pound cover card stock (Hamilco; Denton MD), Southworth 100% cotton thesis paper 20;b/75 GSM (Neenah, Inc; Atlanta GA) or Savoy 100% cotton paper 118 GSM (32/80 lb text). Other suitable materials include blotter paper, rayon filter paper, blanket qualitative filter paper, Whatman No. 1 paper, Kim-Wipe, Schleicher & Schuell filter and electrophoresis paper products such as SS-598, 2043a and 593, glass fiber and non-woven fabric such as bonded polyester or bonded nylon and other filter paper types.

In one embodiment, the thickness of the absorbent material is determined by the thickness of the paper that is applied to the tab. In another embodiment, the thickness, length and width of the absorbent material are all relatively equal, such that the absorbent material is a cube. The advantage of the cube shape is the ability to hold more nucleic acid molecules, such as a plasmid library of DNA molecules. For example, a card having a cube-shaped absorbent material is able to hold a library of plasmids, cDNAs, or genomic DNA, which can serve as templates for PCR amplification of a single subunit of genetic material from within the library. A cube shape may comprise layers of absorbent paper or may comprise an encapsulated material formed from an absorbent powder. An exemplary cube may comprise absorbent material that is approximately 1 cm³, but cubes of others sizes, larger or smaller, are contemplated.

Adhering other absorbent materials such as an absorbent powder is also contemplated. For example, a matrix of glass silica can form a DNA binding vesicle when encapsulated in a “cage” that can be adhered to the tabs. Cages can be filled with untreated and/or treated materials that bind with nucleic acids. Glass silica is exemplary of a material with intrinsic nucleic acid binding properties, wherein the positively charged silica particles have high affinity for negatively charged nucleic acid backbones. Various support materials, including but not limited to silica, cellulose, agarose, or various plastics in a bead, slurry, or resin format can be treated with compounds to improve or add nucleic acid binding activity. Examples include but are not limited to linking diethylamino ethyl (DEAE) to silica beads or linking arginine ligands to agarose.

There are many adhesives known in the art that are suitable for applying the absorbent material to the underlying substrate of the card without affecting the integrity of nucleic acids applied to the absorbent material. The adhesive should be non-toxic since a typical use for eluted nucleic acids is transforming bacteria or eukaryotic cells.

In one embodiment, the adhesive is a double-sided tape with pressure-sensitive adhesive exposed on both sides, allowing two parts to be bonded together by the carrier tape between them. A carrier that holds adhesive can range from a film as thin as a fraction of a millimeter up to a thick foam that helps damp vibrations. Similarly, adhesives can range from low-tack that allows for repositioning all the way up to a permanent bonding solutions. A double-sided tape that has a carrier can be produced with the same adhesive on both sides, or with different adhesives to meet the bonding requirements of different substrates. A suitable adhesive typically is one that adheres well to plastic and/or metal. An example of a suitable double-sided tape is 3M™ Double Coated Tape 9495LE (3M Company, St Paul MN). Other examples of suitable adhesive double-sided tapes include but are not limited to proprietary adhesive tapes produced by the 3M Company having item numbers 9310LE, 93015LE, 415, 93005LE, 9471LEm 9457, 465 and 6035PC. The adhesive on the tape may comprise any type of polyester fibers, such as acrylic or polyvinyl acetate. Other adhesive types may be wet, contact, reactive, single-component reactive, two-component reactive, thermosetting, hot-melt or pressure sensitive adhesives. For example, the adhesive may be a liquid that is sprayed, painted or otherwise spread on the substrate, or it may be a solid, such as the double-sided tapes. Adhesives known as “superglue”, comprising cyanoacrylate esters or similar polymers may be used. The use of epoxy, polyurethane, polyimide adhesives are also contemplated.

Attachment of the absorbent material to the support may be accomplished in a variety of ways. A suitable way is by use of a double-faced adhesive material. The adhesive material is layered down onto the support with tape liner on top. The aperture may then be punched out, the tape liner removed, and the carrier affixed by the adhesive surrounding the aperture. Other suitable methods for attaching the carrier to the support include heat sealing and ultrasonic sealing. Still another method is to dispose the carrier between two supports provided with apertures generally in line with each other. It will be understood that the method of attachment is not limited to the methods described, since any non-toxic adhesive may be used according to manufacturer's instructions or any other method that accomplishes attachment of the absorbent material to the substrate.

Nucleic acids are typically stored in a clear buffered solution, such as 1×TE buffer. A liquid dye may be added to an aqueous solution of nucleic acids to provide a visual indication that the solution has been spotted onto a tab. Neutral dyes are known in the art and are used to visualize DNA or RNA samples for loading electrophoretic gels, including but not limited to bromophenol blue, xylene cyanol and cresol red. Proprietary dyes are also known, including SYBR™ Safe (Invitrogen; Waltham MA), EvaGreen® (Biotium; San Francisco CA) and others may also be used, however, inexpensive neutral loading dyes are generally sufficient. By adding a neutral dye to the nucleic acid solution prior to spotting, a human or a robotic optical system can confirm that any and all tabs have been spotted by visualizing the colorized dye. Additionally, the movement of the dye into an elution solution can be observed and provides an indication that the desired nucleic acids have eluted off a tab.

The invention is particularly well-suited for storing small aliquots of nucleic acids for an extended period of time, such as weeks, months or even years. Thus, another embodiment is a method of using the card for nucleic acid storage by spotting an aqueous solution of nucleic acids onto each individual tab for storage and/or distribution. The aqueous solution of nucleic acids may comprise a dye that will color the absorbent material when the solution has been applied and dried. After drying, the card may be scaled in a light-blocking envelope or flat bag for storage. Storage may be at an ambient temperature, under refrigeration or in a freezer.

In another embodiment, the invention is a method of distributing nucleic acid samples, such as sending the nucleic acids to a colleague or a customer who wished to purchase the nucleic acids. In addition to the labels on individual tabs, the card may have an identifying label, such as a number, a QR code, a bar code, or other type of label. A label may identify the card as comprising a group of nucleic acid types, such as plasmid DNA vectors with various inserts that are individually spotted and labeled on the tabs. A sealed envelope or flat bag containing the card may be distributed to an end user by mail or any other conventional means of shipping. To recover the nucleic acids from the card, an end user can break or cut one or more tabs from the card. A tab is inserted into a microtube having a desired volume of an elution fluid with the distal end in contact with the elution fluid to allow the nucleic acids to transfer from the absorbent material into the elution fluid. The distal end of each tab may be curved or pointed to allow the absorbent material to be fully submerged in the elution fluid.

A card may have tabs wherein each tab holds 2 or more plasmid types, and may hold as many as 3, 4, 5, 6, 7, 8, 9, 10, 11, 12 or more plasmid types. A tab holding a plurality of plasmid types is especially well-suited as a pool of templates for PCR amplification since specificity of PCR primers will determine the identity of an amplification product. While a cube may be able to store 1,000 or more plasmids or other nucleic acid libraries, any embodiment of the card is able to store a plurality of nucleic acid molecules. In one embodiment, a cube contains 500 unique plasmid DNA molecules. An accompanying card has tabs which each have adsorbed to them unique PCR Primer Pairs. These PCR primer pairs can be used to amplify a specific genetic sequence found on one of the 500 plasmids on the cube.

Before exemplary embodiments of the present invention are described in greater detail, it is to be understood that this invention is not limited to any particular embodiments described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

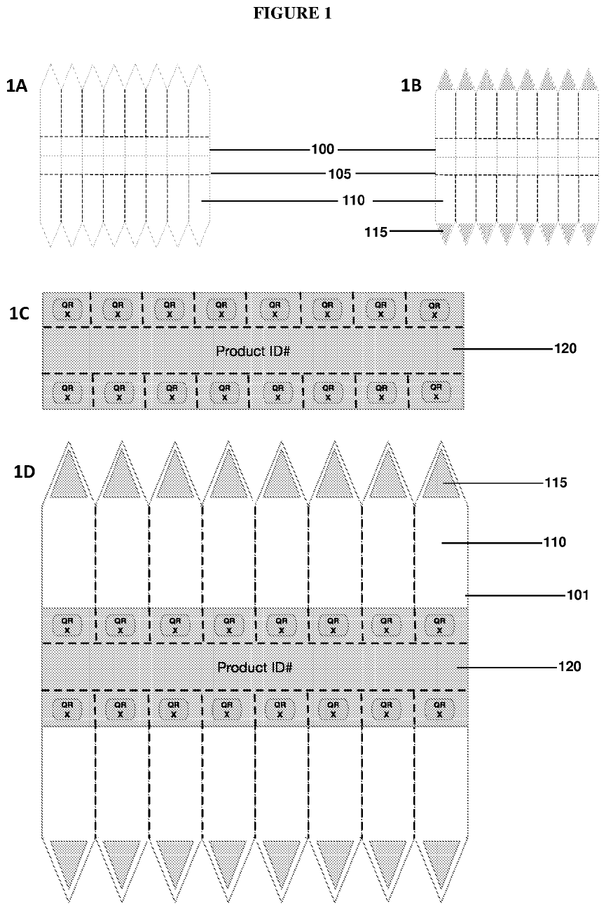

The following Examples provide exemplary compositions and methods for making and using the nucleic acid storage card of the invention. These Examples describe materials and methods for using embodiments illustrated in FIGS. 1-7. Additional details can be found in the section entitled “Brief Description of the Drawings”.

### Example 1

### Components of a Nucleic Acid Storage Card

FIG. 1A illustrates a card 100 having detachable tabs 110 defined by lines 105 that may be printed and cut with scissors or perforated and broken or snapped to remove a tab of interest. The distal ends of the tabs are cut to form points. In this exemplary embodiment, the card consists of 16 tabs. FIG. 1B shows distal ends of the tabs having a pointed shape with a triangular piece of absorbent material adhered to each tab. FIG. 1C shows a label 120 that can identify each tab with a unique QR code. The central region of the label 120 can be printed with a label identifying the entire card. FIG. 1D shows an assembled card 101 identified with a product label 120 identifying the card and individual tabs 110 with QR codes. The unique QR code for each tab links to a website with information about the nucleic acid molecules spotted on that tab. The information can include but is not limited to an RNA or DNA map, a sequence file, links to reference materials, a description of various elements such as regulatory elements, promoter identification, insert identification, location and sequences of primers, restriction enzyme sites and antibiotic resistance gene(s) for selective growth on a culture plate or in liquid culture.

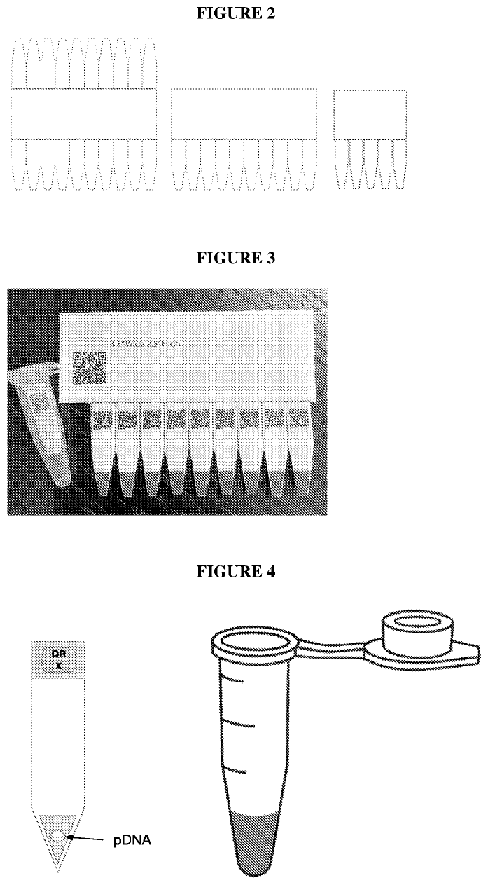

FIG. 2 shows line drawings of cards having various numbers of detachable tabs, including 20, 10 or 5.

### Example 2

### Use of a Neutral Dye to Indicate the Presence of Nucleic Acids

FIG. 3 illustrates a card comprising 10 detachable tabs that were spotted with a nucleic acid solution that contained a neutral blue dye. One tab has been detached and placed inside a microcentrifuge tube and is ready for addition of an elution fluid.

### Example 3

Transfer of Nucleic Acids from a Tab to an Aqueous Solution

Each portion of absorbent material can be spotted with a different sample of nucleic acid molecules, such as a plasmid vector carrying a recombinant DNA insert or clone. FIG. 4 shows a detached tab that was previously spotted with an aqueous solution of plasmid DNA with a neutral blue dye. The QR code is carried with the tab so that an end user can identify the plasmid DNA. The detached tab is placed into an elution buffer in the microtube. The blue neutral dye provides a visual indicator of transfer from the absorbent material into the elution buffer. After elution, the tab is removed and discarded. The label on the tab is a plastic sticker that can be peeled off the tab before discarding and placed on the microtube to carry the identification with the eluted plasmid DNA.

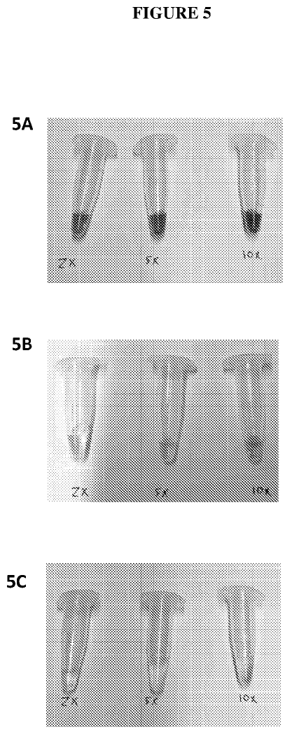

FIG. 5 illustrates the concomitant transfer of a neutral dye along with plasmid DNA that were spotted on tabs of a card in concentrations of 2×, 5× or 10×, with 1× equal to 1 μl of dye added to 50 μl of TE buffer. 5A shows xylene cyanol, 5B shows bromophenol blue, and 5C shows cresol red.

### Example 4

Integrity of Nucleic Acids Eluted from a Tab

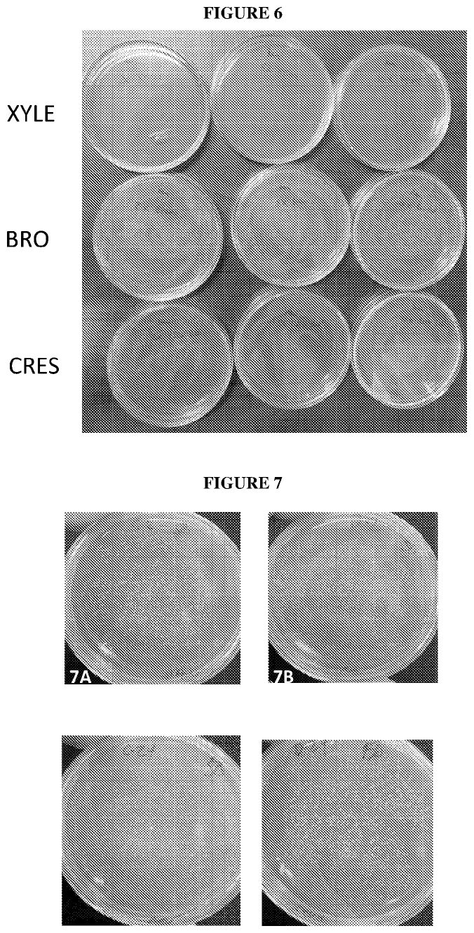

FIG. 6 shows growth of bacteria transformed with the kanamycin-resistant plasmid DNA eluted in FIG. 5 and plated on agar plates with ampicillin. Xylene cyanol is toxic to the bacteria, and no colonies were observed. Growth was abundant on plates with bacteria transformed with plasmid DNA recovered from tabs spotted with solutions comprising bromophenol blue or cresol red, indicating that the plasmids were intact, and the dye was a suitably non-toxic.

### Example 5

### Test of the Effects of Various Adhesives on Nucleic Acid Integrity and Bacterial Toxicity

Some adhesives can damage nucleic acids or may contain components that are toxic to bacteria. FIG. 7 shows growth of bacteria transformed with kanamycin-resistant plasmid DNA that was spotted on absorbent material adhered to the plastic substrate with various adhesives. The adhesives used were: 7A: 3M™ 9495LE, 7B: 3M™ 93010LE, 7C: 3M™ 93015LE and 7D: 3M™ 415. Aliquots of kanamycin-resistant plasmid DNA suspended in TE buffer were spotted on tabs, dried overnight, and eluted from the absorbent material. The eluents were used to transform E. coli and spread on agar plates comprising ampicillin. All four plates show good recovery of the plasmid DNA, with abundant growth indicating integrity of the nucleic acids and no toxic contaminants carried over from the adhesives.

### Example 6

### High-Speed Production and Use of Card

Card backer material having a thickness of 20 0 mil is used to form a quantity of cards using an automated system that prints, treats, and feeds through an attachment station of filter paper. Cards are then cut out to desired size and shape for various applications. Each card produced can have multiple styles from perforated tabs to self-snipping or snap/tear away creating a way for the user to remove each tab.

DNA plasmids are spotted on the tabs, with one plasmid species per tab. A label may be printed on each card during the production process. Alternatively, the cards are “blanks” that are produced without a label, and special peel-and-stick labels are applied to the cards at a later time. The label is used to identify the card type, i.e., to identify that each is carrying a collection of specific plasmids. A secondary label printed or applied on each tab in the form of a QR code identifies the specific plasmid that was spotted an individual tab. Each QR code encodes a link that provides an annotated map for the related DNA plasmid and instructions for elution and propagation or the user's specified link to data.

The cards are cured and individually may be sealed inside a mylar envelope that is also labeled to identify the card type and plasmids. The packaged cards are stored at room temperature or other controlled environment until mailed or shipped to an end user. The user is able to identify the card and the plasmids using the labels and QR codes with instructions for use.

### Example 7

### Storage of PCR Primers on a Card

The cards are useful for storing oligonucleotides, such as those used as PCR primers. The oligonucleotides may be stored as a single oligonucleotide type (i.e., a single forward or reverse primer), in pairs (i.e., a forward and reverse primer pair for a specific template), or in groups of pairs (i.e., forward and reverse primer pairs for various templates). Furthermore, they may be stored on a single tab, or as an array of singles, pairs, or multiples.

### Example 8

### Amplification of Template Libraries Stored on a Card Using Primers Stored on a Card

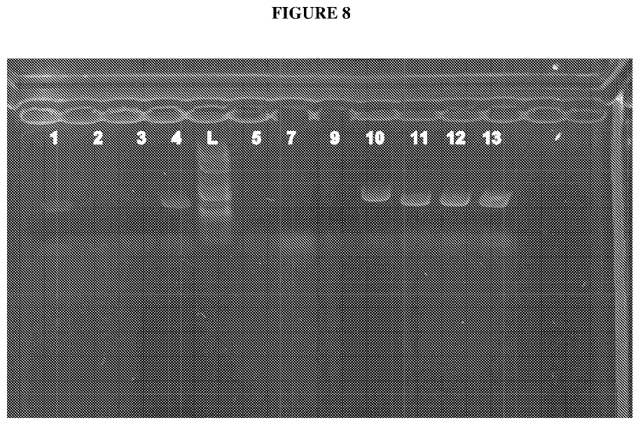

Five distinct pDNA species were spotted and stored on individual tabs on a card as single templates. A group of all 5 DNA species was spotted and stored on a sixth tab. Specific primer pairs were synthesized and spotted as pairs on another card. The cards were stored at ambient temperature for a period of 14 days and the contents of each tab were eluted in 100 microliters of TE buffer. PCR amplification of various combinations of the eluted templates and primer pairs was performed and samples of the combination shown in Table 1 were electrophoresed in an agarose gel, as shown in FIG. 8. Appropriate amplification was demonstrated in lanes 1 and 4 for DNA1 and DNA2, as well as a combination of DNA1 and DNA2 in lane 10 and a combination of all five templates, DNA1-DNA5, in lanes 11-13. This example demonstrates safe storage and maintenance of integrity of the reactants.

TABLE 1
Combinations of template DNA and primer
pairs for PCR amplification

| | Template | Primer | | Template | Primer |
|---|---|---|---|---|---|
| Lane | DNA | Pairs | Lane | DNA | Pairs |
| 1 | DNA1 | P1 | 7 | DNA5 | P7 |
| 2 | DNA1 | P2 | 9 | DNA1 + DNA2 | P2 |
| 3 | DNA2 | P3 | 10 | DNA1 + DNA2 | P3 |
| 4 | DNA2 | P4 | 11 | DNA1 − DNA5 | P4 |
| 5 | DNA3 | P5 | 12 | DNA1 − DNA5 | P1 |
| | DNA4 | | 13 | DNA1 − DNA5 | P4 |
| 7 | DNA5 | P7 | 7 | DNA5 | P7 |

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein

We claim:

1. A card for storage of nucleic acid molecules, comprising
a flat solid substrate having perforations forming a plurality of detachable tabs,
an absorbent material adhered to an upper surface or bottom surface of a distal end of each of the detachable tabs, and
a proximal region on each of the detachable tabs wherein a label may be applied.

2. The card of claim 1, wherein the flat solid substrate is selected from the group consisting of plastic (e.g., polyethylene (PET), polyvinylchloride (PVC), polyethylene terephthalate (PETG), polyimide, polycarbonate, polystyrene) ceramic, glass, paper or plastic-paper laminate or a combination of any of these.

3. The card of claim 3, wherein the absorbent material prior to adhering to the upper surface or the bottom surface is treated with at least one agent having nucleic acid-protective properties.

4. The card of claim 3, wherein the at least one agent is selected from the group consisting of a desiccant, a nuclease inhibitor and an ion chelator.

5. The card of claim 1, wherein the absorbent material is a porous paper.

6. The card of claim 1, wherein the absorbent material is adhered with an adhesive.

7. The card of claim 6, wherein the adhesive comprises a pressure-sensitive double-sided tape.

8. The card of claim 1, wherein the label is applied by printing or application of an identifying sticker.

9. The card of claim 6, wherein the label identifies the nucleic acid molecule absorbed by the absorbent material.

10. The card of claim 1, wherein one or more nucleic acid molecules is absorbed on the absorbent material of one or more of the detachable tabs.

11. A method for storage and/or distribution of nucleic acid molecules to an end user, comprising:
providing a card comprising a flat solid substrate having at least one detachable tab with absorbent material adhered to an upper surface or bottom surface of a distal end of the at least one detachable tab;
applying an aqueous solution comprising one or more nucleic acid molecules to the absorbent material on the at least one detachable tab;
allowing the aqueous solution to dry;
printing or applying a label identifying the nucleic acid molecules applied to the at least one detachable tab;
sealing the card in packaging; and
storing the packaged card under suitable conditions and/or distributing the packaged card to an end user.

12. The method of claim 11, wherein the suitable conditions are selected from the group consisting of room temperature, refrigeration and freezing.

13. The method of claim 11, further comprising mailing or shipping the packaged card to the end user.

14. A card for storage of a library of nucleic acid molecules, comprising
a flat solid substrate,
a distal end having a cube of absorbent material adhered to the substrate, and
a proximal end having a label applied to the substrate.

15. The card of claim 14, wherein a library of nucleic acid molecules is absorbed on the cube of absorbent material.
